(19) United States
(12) Patent Application Publication
(10) Pub. No.: US 2008/0119782 A1
(43) Pub. Date: May 22, 2008

(54) METHOD FOR DELIVERING SOLUTIONS TO A PATIENT

(76) Inventors: Christopher P. Steinman, Sandy, UT (US); Jason A. Bultman, Salt Lake City, UT (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(21) Appl. No.: 11/875,791

(22) Filed: Oct. 19, 2007

Related U.S. Application Data
(60) Provisional application No. 60/862,919, filed on Oct. 25, 2006.

(51) Int. Cl. A61M 1/00

(52) U.S. Cl. 604/28

(57) ABSTRACT

A method for infusing solutions utilizes a solution reservoir divided into a first section and a second section which are fluidly isolated but volumetrically connected such that introduction of a pumping fluid into the first section of the reservoir causes displacement and flow of solution out of the second section of the reservoir. The use of a separate reservoir, separation member, and delivery tubing allow for significantly decreased loss of feeding solution due to dead volume. The solution reservoir can be configured to provide proportionally different flow.

### RELATED APPLICATIONS

The present application claims the benefit of U.S. Provisional Patent Application No. 60/862,919, filed Oct. 25, 2006, which is expressly incorporated herein.

### BACKGROUND OF THE INVENTION

1. The Field of the Invention

The present invention relates to the feeding of patients that may benefit by the reduction of lost or wasted feeding solution including but not limited to the neonate, pediatric, or similar patient population. More specifically, the present invention relates to a system for neonatal feeding which uses an intermediate feeding reservoir to provide improved feeding and reduced waste.

2. State of the Art

Many infants which are born prematurely or which are smaller or underdeveloped do not have sufficient mouth strength to feed normally. That is to say that many premature infants do not have sufficient strength to breast feed or to draw milk from a bottle. These infants are typically fed using a feeding pump which delivers the milk, etc. through a nasal feeding tube which has been placed through the nose or through an enteral feeding tube placed in a stoma in the stomach wall. Due to their size, neonatal infants may require very slow administration of feeding solution, on the order of 1 mL per hour.

Several limitations may arise in using these feeding systems. One limitation is that the mothers of premature infants may not be producing a large quantity of milk. Another limitation is that some of the feeding solution is often lost as waste in the tubing and reservoirs of the feeding system when the disposable set is discarded. Yet another limitation is that the milk may tend to settle as it is stored in the reservoir awaiting delivery to the infant. As the rate of feeding may be very slow, there is sufficient time for the solution to separate into its water and fat components before delivery to the infant. Another problem is that the solution in the feeding pump may get cold during delivery.

It is thus desirable to provide an improved feeding system which overcomes these and other limitations of available feeding systems. Such a feeding system should allow for convenient and safe delivery of the solution, should provide improved delivery conditions, and should reduce waste of the feeding solution, helping to improve patient outcomes.

Other situations exist where it is similarly desirable to provide a solution to a patient in a slow and controlled manner, while keeping the solution mixed, while keeping the solution temperature controlled, while reducing the volume of lost solution, etc. Such solutions may include medicament solutions, IV solutions, etc. For brevity, feeding solutions such as milk, formula, enteral feeding solutions, medicament and IV solutions are collectively referred to as medical solutions or feeding solutions. There is a need to provide such solutions to a patient in a more controlled fashion, such as by controlling the delivery rate, separation of the solution, temperature of the solution, etc.

### SUMMARY OF THE INVENTION

It is an object of the present invention to provide an improved delivery system for enteral feeding.

According to one aspect of the present invention, a system is provided which reduces the amount of medical solution which is wasted. The system may utilize an intermediate reservoir which holds the solution and a pumping reservoir which holds another solution, such as water. A pump may be used to pump the water and thereby displace the delivered solution from the intermediate reservoir, while maintaining the volume and flow rate delivered by the pump.

According to another aspect of the present invention, a system is provided which may warm or cool the solution during delivery. An intermediate reservoir may be used to contain and deliver the medical solution, and may be placed in a temperature controlled bath or other heating means to warm/cool the solution. A reservoir separate from the pump may be more easily heated than a reservoir attached to the pump.

According to another aspect of the present invention, a system is provided which may efficiently mix the medical solution during delivery to prevent separation of the solution. An intermediate reservoir may easily be placed in an agitator or other mixer during delivery to prevent separation of the medical. Because the agitator, etc. may be placed closer to patient than the pump, the medical, such as milk has less opportunity to separate than if the milk is in a reservoir upstream from the pump.

These and other aspects of the present invention are realized in a feeding system as shown and described in the following figures and related description.

### BRIEF DESCRIPTION OF THE DRAWINGS

Various embodiments of the present invention are shown and described in reference to the numbered drawings wherein:

FIG. 1 shows a schematic view of the system of the present invention;

FIG. 2 shows a perspective view of a pump fluid reservoir of the present invention;

FIGS. 3A and 3B show perspective views of prior art pumps as may be used with the present invention;

FIGS. 4A and 4B show solution reservoirs of the present invention;

FIG. 5A shows a side view of another solution reservoir of the present invention;

FIG. 5B shows a side view of a prior art breast pump as may be used with the reservoir of FIG. 5A and with the present invention;

FIG. 5C shows a side view of a dispensing cap for use with the reservoir of FIG. 5A;

FIG. 6A shows a side view of another solution reservoir of the present invention;

FIG. 6B shows a side view of a dispensing cap for use with the reservoir of FIG. 6A; and

FIG. 7 shows a perspective view of the system of the present invention.

It will be appreciated that the drawings are illustrative and not limiting of the scope of the invention which is defined by the appended claims. It is further appreciated that not all aspects or structures of the invention may be shown in a single drawing, and as such various drawings illustrate smaller parts of the invention shown in other drawings. The various embodiments shown accomplish various aspects and objects of the invention.

### DETAILED DESCRIPTION

The drawings will now be discussed in reference to the numerals provided therein so as to enable one skilled in the art to practice the present invention. The drawings and descriptions are exemplary of various aspects of the invention and are not intended to narrow the scope of the appended claims. It is appreciated that not all structures and elements of the invention may be shown in a single drawing and multiple drawings are therefore presented, each drawing more clearly illustrating all or a portion of the invention.

The present application discusses the invention in the context of delivering a feeding medical to an infant. It is appreciated that there are other uses for the present system, such as delivering medication, nutrition solutions, IV solutions, etc. to a patient. Delivery of such solutions is typically done by the use of tubing which is attached to a reservoir for holding the solution and a pump for providing a controlled delivery of the solution. Such tubing is commonly referred to as an infusion set, and is used herein to reference the tubing and related structures used for the delivery of food, IV solutions and other solutions to a patient. The present invention provides an advantageous system for accomplishing these uses, by lowering wasted medical, controlling the temperature of the medical, improving accuracy in the delivery rate or quantity of the medical, etc.

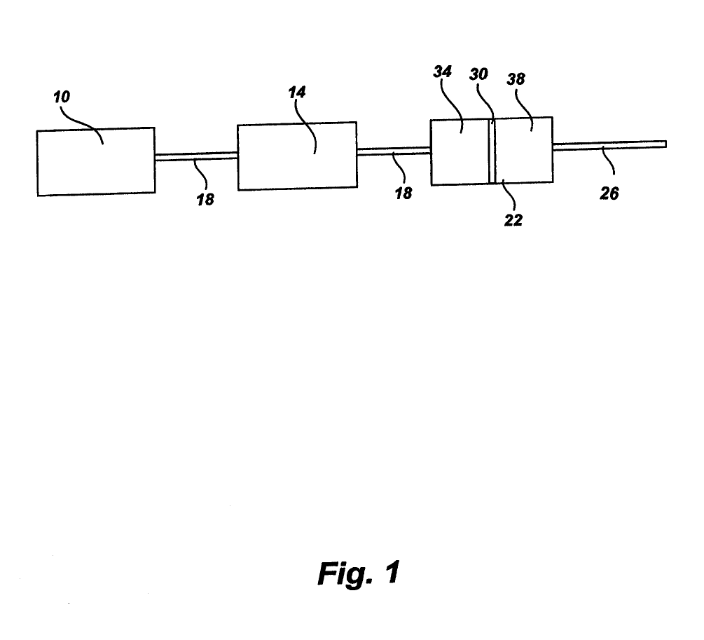

Turning now to FIG. 1, a schematic diagram of a feeding system of the present invention is shown. The feeding system typically includes a pump fluid reservoir 10, a pump 14, a pump tubing 18, a solution (patient nutrition, etc.) reservoir 22, and a patient delivery tubing 26 consistent with a conventional infusion set and pump. The delivery system utilizes a solution reservoir 22 disposed along the infusion set which includes a separation member 30, such as a diaphragm, piston, movable wall, or plunger, to separate the reservoir into two sections. A first section 34 is configured to receive a pump fluid such as water. A second section 38 contains the medical solution, typically milk. The solution reservoir 22 and separation member 30 allow for a changeable distribution of volume between the first section 34 and the second section 38, and is designed to expel all or nearly all of a patient nutrition solution to the patient delivery tubing. That is to say that the solution reservoir 22 and separation member 30 are designed such that pumping a volume of pump fluid into the first section 34 displaces a volume of feeding solution from the second section 38. The solution reservoir, as will be illustrated, can be designed to deliver various ratios of feeding solution to pump fluid, such as 2:1, 1:1, 1:2, etc.

Thus, in use, the second section 38 of the solution reservoir 22 is filled with feeding solution. The second section 38 is connected to the delivery tubing 26 such that when feeding solution exits the second section, it exits via the delivery tubing. A pump fluid reservoir 10 is filled with a pump fluid, typically water, saline, or an inexpensive and available liquid. The pump fluid reservoir 10 is connected to a pump tubing 18. The pump tubing carries the pump fluid through the pump and to the first section 34 of the solution reservoir 22 in the case of a peristaltic pump. In the case of a syringe pump, the pump applies force to the pump fluid reservoir and thus drives solution through the pump tubing 18.

It will be appreciated that the exact nature of the pump tubing 18 will depend on the design of the pump 14. Various types of pumps are suitable for the present invention. If the pump 14 has an inlet connection and an outlet connection, the pump tubing 18 may comprise two pieces; one piece to connect the pump fluid reservoir 10 to the pump inlet and a second piece to connect the pump outlet to the first section 34 of the solution reservoir 22. If the pump 14 is a peristaltic pump, the pump tubing 18 may be a simple piece of tubing which engages the pump drive (fingers or rollers, typically) and connects to the pump fluid reservoir 10 and to the solution reservoir 22. Alternatively, the pump tubing 18 may be a pump cartridge configured to work with a particular model of pump. Such a pump cartridge may include an inlet tubing, outlet tubing, pump engaging tubing, connectors, etc. Such cartridges or pump tubing are known in the art and will be selected according to the desired pump 14.

In operation, an operator fills the second section 38 of the solution reservoir 22 with feeding solution, such as breast milk, and connects the second section of the solution reservoir to a delivery tubing 26. The operator then connects the pump fluid reservoir 10 full of pump fluid to the pump tubing 18. The pump tubing 18 is connected to or routed through the pump 14 as may be necessary. The pump 14 is then primed (operated so as to move fluid through the pump tubing 18) to remove air from the pump tubing 18 and pump fluid reservoir 10, and the pump tubing 18 is then connected to the first section 34 of the solution reservoir 22. It is appreciated that the various steps of operating the system may be changed somewhat as to order or operation according to the pump used or to the desired order of operation. For example, the pump may be first primed and prepared and the feeding solution later prepared and filled into the solution reservoir 22.

The pump 14 may then again be primed to force pump fluid into the first section 34 of the reservoir 22, and also to force any air out of the second section 38 of the feeding reservoir and delivery tubing 26 if desired. If desired a bleed valve or the like may be provided, or, for simplicity, the air may simply be pumped out of the delivery tubing prior to connecting the tubing to the patient. The pump 14 may then be programmed for the desired dosage volume and flow rate. The delivery tubing 26 is connected to the patient, often an infant, and the pump is operated to deliver the feeding solution to the patient.

In order to deliver the feeding solution to the patient, the pump 14 moves the selected flow rate and volume of pump fluid from the pump fluid reservoir 10 to the first section 34 of the solution reservoir 22. The flow of pump fluid into the first section 34 of the solution reservoir 22 displaces an equal volume of feeding solution from the second section 38 of the solution reservoir, and thereby delivers the selected flow rate of feeding solution to the patient.

The prior art devices allow for feeding of an infant or other patient, but suffer from several disadvantages. For example, many prior art devices hold the feeding solution in a pump-mounted reservoir. Such a reservoir can not easily be agitated or heated/cooled. Additionally, prior art devices move the feeding solution through the pump and to the patient. As such, a significant volume of feeding solution can remain unusable in the tubing and delivery set, and in the pump reservoir. Many mothers of premature infants are not producing a high quantity of milk, and loss of even several milliliters of the milk is significant.

The present system overcomes these and other limitations by providing a separate solution reservoir 22 that connects to a short delivery tubing 26 or patient feeding tube through which the feeding solution is delivered. The following figures show and discuss the various sub-structures of the present invention so as to further explain the system of FIG. 1.

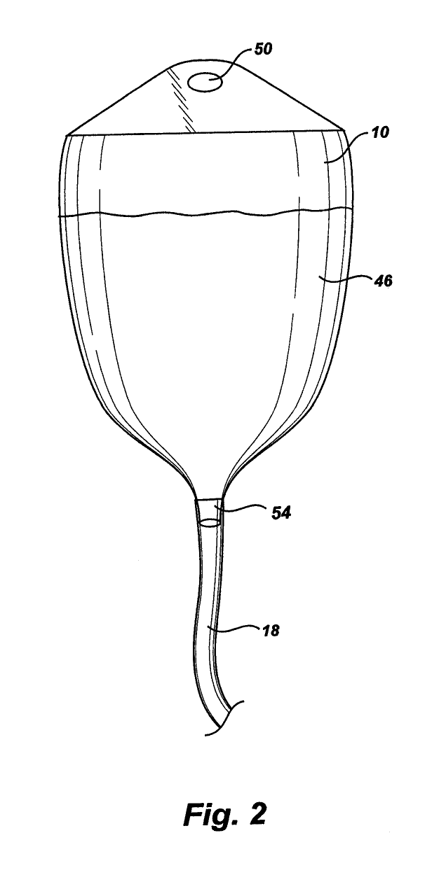

Turning now to FIG. 2, a perspective view of a pump fluid reservoir of the present invention is shown. The pump fluid reservoir 10 should hold a sufficient amount of pump fluid 46 for the amount of feeding solution delivered to the patient. In the case of infants, the amount of fluid is typically small, and the reservoir 10 need not be overly large. An IV bag or a conventional feeding bag may be an ideal reservoir. The reservoir 10 may optimally have an opening 50 which may be used to hang the reservoir when in use. The pump fluid reservoir 10 may have a connector 54 to allow for connection to the pump tubing 18.

The pump fluid 46 may commonly be water, saline, or another commonly available fluid. It may be desirable that the pump fluid 46 be non-toxic such that no harm results if pump fluid is accidentally introduced into the feeding solution. The pump fluid reservoir 10 should preferably provide a steady, unrestricted, and uninterrupted flow of pump fluid 46 to the pump. It is generally not desirable that the flow be interrupted, or that air is introduced into the pump tubing 18. However, such configurations are not excluded from the present invention.

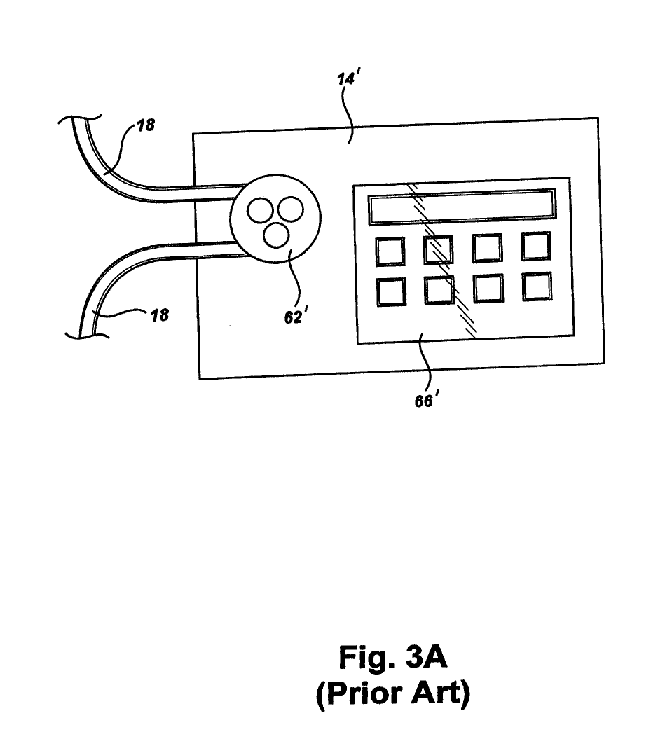

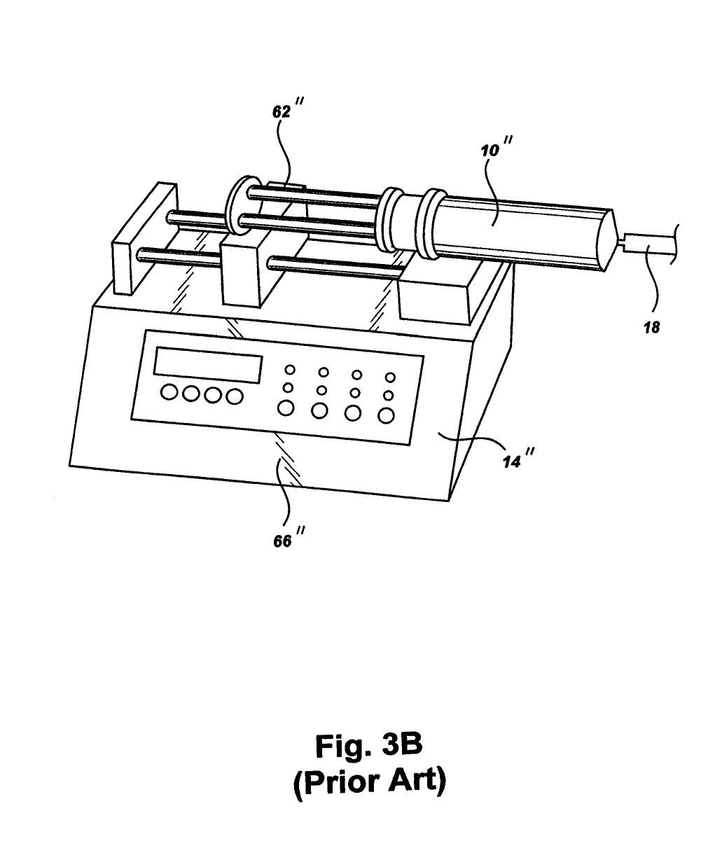

Turning now to FIG. 3A, a perspective view of a prior art pump as may be used with the present invention is shown. The pump 14′ may be many different types of pumps, such as a rotary peristaltic pump, linear peristaltic pump, syringe pump, piston pump, etc. Thus, any of the pumps may be used as pump 14 in FIG. 1. A currently preferred type of pump is a peristaltic pump. As such, the pump 14′ may have a pumping mechanism 62′ such as a rotor, or may alternatively have a linear peristaltic actuator, piston, etc. For such a peristaltic pump 14′, the pump tubing 18 may simply wrap around the pumping mechanism (rotor) 62′ and be anchored into position in the pump. Alternatively, the pump tubing 18 may be part of a pump cartridge, or may include various tubing sections such as inlet tubing, outlet tubing, pump rotor engaging tubing, etc. and the various joints or connectors necessary for operation.

The pump 14′ may also include a control panel 66′ and associated control circuitry for controlling the operation of the pump. Such is desirable as it allows the user to start and stop the pump, prime the pump, set the flow rate and delivered volume, etc. It is desirable that the pump 14′ which is used have control circuitry as is common to feeding pumps so as to provide the same functionality and safety features when used with the solution reservoir of the present system.

Turning now to FIG. 3B, a perspective view of another prior art pump as may be used with the present invention is shown. A syringe pump 14″ is shown. The syringe pump 14″ includes a pumping mechanism 62″ in the form of a linear actuator which moves a syringe piston. With such a pump, a syringe forms the pump fluid reservoir 10″. The syringe/pump fluid reservoir 10″ is attached to the pump tubing 18 which carries the pump fluid to the solution reservoir 22. The pump fluid is pumped into the first section 34 of the solution reservoir 22 as discussed previously. The pump 14″ typically includes control means such as control panel 66″. It will be appreciated that it is important to control the operation of the pump sufficiently to provide safe and accurate feeding to an infant. As such, many different types of pumps 14″ may be used with the present system. Thus, any of the pumps and other components discussed herein may be used in the system shown in FIG. 1.

One advantage of the present invention is that it is a relatively inexpensive way to improve feeding of neonatal infants. Prior art pumps can be used, preserving a hospital or other institution's investment in equipment. By providing delivery tubing with an intermediate solution reservoir, only additional feeding sets, reservoirs, or tubing are necessary. As some neonatal pumps cost thousands of dollars, the ability to use existing pumps is a significant cost advantage. Additionally, pump fluid may be reused or inexpensive fluid such as water or saline, providing additional savings.

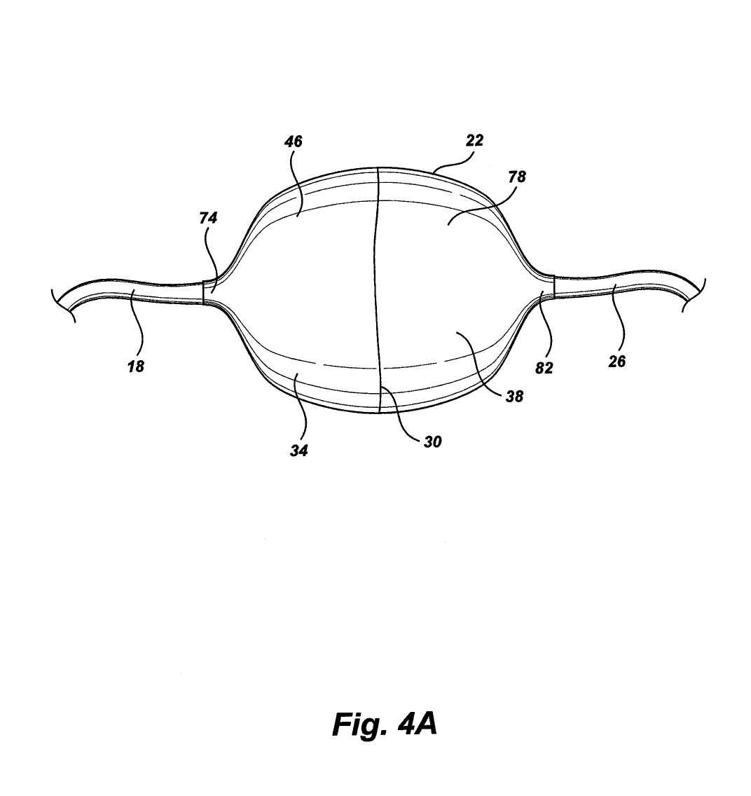

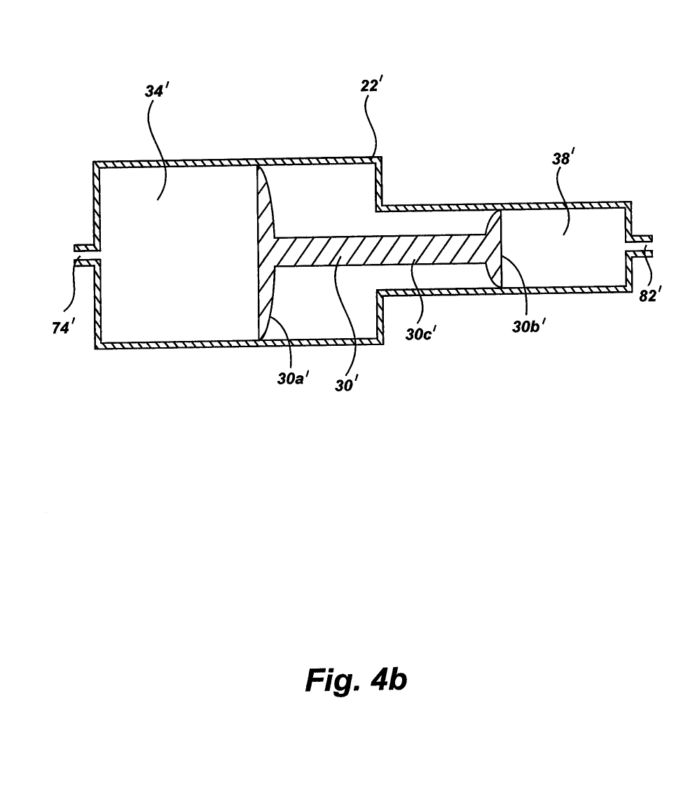

Turning now to FIG. 4A, a side view of a solution reservoir of the present invention is shown. As has been discussed, the solution reservoir 22 uses a separation member 30 to divide the solution reservoir into a first section 34 and a second section 38. The first section 34 receives pump fluid 46 from the pump 14. The first section is thus typically formed with a connector 74 which is used to connect the first section to the pump tubing 18. The second section 38 of the solution reservoir 22 contains the feeding solution 78 which is delivered to a patient. The second section 38 of the solution reservoir 22 is typically constructed with a connector 82 to allow connection of the delivery tubing 26 to the second section. It is appreciated that connectors 74, 82, and others of the present invention may be of many suitable types, including slip fit connectors, locking or twisting connectors such as Luer connectors, etc.

The separation member 30 is designed such that fluid flow between the first section 34 and the second section 38 is prevented. Thus, the separation member 30 prevents any introduction of the pump fluid 46 into the feeding solution 78. The separation member, however, allows for a variable distribution of volume between the first section 34 and the second section 38 of the solution reservoir 22. Fluid flow into the first section 34 of the solution reservoir 22 may directly displace fluid from the second section 38. The separation member 30 may comprise a slidable piston or moveable plunger in a reservoir such as a cylinder, or may comprise a flexible membrane or diaphragm in a reservoir, such as a cylinder, bag, etc. Thus, when pump fluid 46 is introduced into the first section 34, an amount of feeding solution is expelled (displaced) from the second section 38. Depending on the geometry of the reservoir, an amount of feeding solution may be expelled which is equal to the pump fluid introduced.

It is desirable that the separation member 30 is designed such that all of the feeding solution 78 can be expelled from the second section 38 of the solution reservoir 22. This allows for more complete utilization of the feeding solution, which may be important if the feeding solution is expensive or in short supply, as may be the case with breast milk from a mother with a premature infant. If the separation member 30 is a flexible diaphragm, the separation member may be shaped so as to allow for complete conformation to the remaining interior surface of the second section 38 of the solution reservoir, allowing for complete or near complete delivery of the feeding solution. The separation member may also be slightly elastic to allow for more complete delivery of the feeding solution. It is appreciated that the separation member should provide little enough resistance to movement, etc. so as to not interfere with the pump operation.

Waste of the feeding solution 78 may be further reduced by using a delivery tubing 26 which has a smaller inner diameter or shorter length with a closer proximity of the solution reservoir to the patient, or by directly connecting the solution reservoir to a feeding catheter or ingress port. It is appreciated that a significant volume of milk or other feeding solution can remain in a tube. Pumping tubing for feeding pumps, such as the feeding delivery sets, often has an internal diameter of about 3 millimeters. Such a tubing may have an internal volume of 0.18 mL per inch of tubing. By contrast, a tubing with an internal diameter of about 1.5 millimeters has an internal volume of 0.045 mL per inch of tubing.

It is appreciated that it is often not feasible or not as effective to use smaller tubing as pumping tubing. The present invention allows the most effective size of tubing to be used as pumping tubing, and small bore tubing to be used for delivering the feeding solution, reducing the volume of feeding solution wasted in the tubing. The loss of feeding solution is further reduced by pumping the feeding solution only through the delivery tubing and not through the pump tubing. By way of example, two feet of delivery tubing with a 1.5 mm bore (tubing of the present system through which solution flows) has a volume of 1.1 mL, while four feet of pumping and delivery tubing with a 3 mm bore (pumping and delivery tubing of a conventional feeding set) has a volume of 8.6 mL, a nearly 8 fold increase in the amount of wasted feeding solution. It is thus appreciated that significant reduction in wasted feeding solution occurs with the present invention, improving patient outcomes, especially in situations where feeding solution is limited such as with breast milk. A reduction in waste means that a higher percentage of the total solution is delivered to the patient, which is especially useful when delivering medication, vitamins, or other supplements with the feeding solution.

Turning now to FIG. 4B, a side cross-sectional view of an alternate feeding reservoir of the present invention is shown. The reservoir 22′ includes a first section 34′ and a second section 38′ which are separated by a separation member 30′. The separation member 30′ is shown as a dual piston, a first piston head 30a′ and a second piston head 30b′ forming each end with a rigid member 30c′ between the pistons. The first section 34′ and second section 38′ are of different diameters (i.e. different cross-sectional areas), as are the first piston head 30a′ and the second piston head 30b′. Such an arrangement provides a differential displacement of fluids from the two reservoirs. The ratio of fluid flow into and out of the first section 34′ and second section 38′ is equal to the ratio of the square of diameters or cross-sectional areas of the first piston head 30a′ and second piston head 30b′. Thus, by adjusting the diameter or cross-sectional surface areas of the piston heads, the delivery can be proportional to the pumped solution, either an equal quantity or some fraction thereof. Thus, depending on the desired flow rate, the first piston head may be larger or smaller than the second piston head.

Each reservoir is typically provided with a connector 74′, 82′. In use, if the first section 34′ had a diameter twice that of the second section 38′, a four to one volume displacement ratio would exist between the sections. Thus, if the first section 34′ were connected to a pump and the second section 38′ were filled with feeding solution and connected to the patient, 1 mL of feeding solution would be delivered to the patient for every 4 mL of pump fluid introduced into the first section 34′ by the pump. Alternatively, if the second section 38′ were connected to the pump and the first section 34′ were filled with feeding solution and connected to the patient, 4 mL of feeding solution would be displaced for every 1 mL of pump fluid introduced. This allows much greater control over the volume of the solution delivered to a patient over a given amount of time. Those skilled in the art will appreciate that this is particularly important when working with infants.

It is thus appreciated that such a reservoir 22′ could be used to multiply or divide the pump output to control the delivery rate to a patient. For example, such a reservoir 22′ could be used to decrease the rate and increase the accuracy of feeding solution delivery to a premature infant. A pump which was capable of delivering 1 mL per minute of pump fluid could be used to accurately deliver 0.25 mL per minute of feeding solution to the infant. Such a reservoir 22′ could be made in different volumes and with different piston area ratios to provide solution as desired. Reservoirs may be made with different displacement ratios such as 1:1, 2:1, 5:1, 10:1, etc. to allow for maximum flexibility in providing solution delivery rates with available pumps.

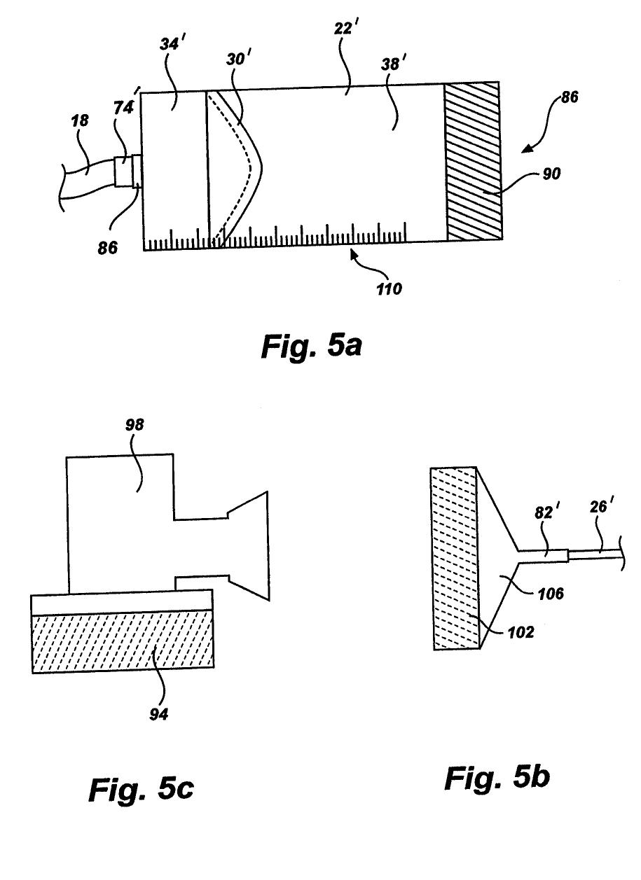

Turning now to FIG. 5A, a side view of an alternative solution reservoir of the present invention is shown. The solution reservoir 22′ includes a piston type separation member 30′ which separates the reservoir into a first section 34′ and a second section 38′. A connector 74′ is typically provided on the first section to allow for connection to the pump tubing 18. A valve 86 may be used in combination with the connector 74′ if such is necessary for the proper operation of a breast pump. The second section 38′ of the reservoir 22′ is typically provided with an open end 86 with threads 90. The threads 90 allow the reservoir to be attached to the threads 94 of a conventional breast pump 98 (FIG. 5C), or to the threads 102 of a dispensing cap 106 (FIG. 5B). The dispensing cap 106 may also snap onto the reservoir 22′. The solution reservoir 22′ may also be graduated 110 to allow for easy measuring of the feeding solution contained in the reservoir.

The dispensing cap shown in FIG. 5B 106 is typically formed with a connector 82′ which allows for connection to the delivery tubing 26. The plunger separation member 30′ is typically formed with a shape corresponding to the inside shape of the dispensing cap 106 to allow all or nearly all of the feeding solution to be dispensed. The separation member may be formed from a flexible material to allow for some conformation to the inside of the dispensing cap 106 to achieve complete dispensing of the feeding solution.

The solution reservoir 22′ as shown is typically used with a breast pump 98, as shown in FIG. 5C. The reservoir 22′ is connected to a breast pump 98 and the second section 38′ is filled with milk. The reservoir 22′ may then be disconnected from the breast pump and connected to a dispensing cap 106. The reservoir 22′ is then used as has been discussed above by priming and preparing a pump and pump fluid reservoir, connecting the solution reservoir 22′, priming, and running the pump to dispense the milk or feeding solution. As pump fluid is introduced into the first section 34′ of the reservoir 22′, the piston separation member 30′ is moved and forces feeding solution from the second section 38′ of the solution reservoir.

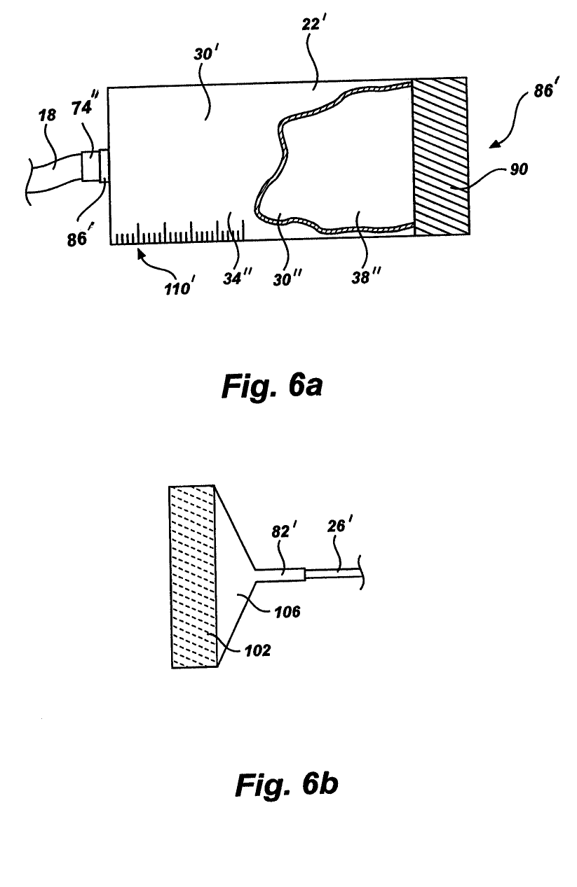

Turning now to FIG. 6A, a perspective view of an alternate solution reservoir of the present invention is shown. The solution reservoir 22″ is similar to that shown in FIG. 5, but utilizes a flexible bag instead of a plunger for a separation member 30″. The bag 30″ fits inside of a container 118 to form the solution reservoir 22″ with a first section 34″ and a second section 38″. A dispensing cap 106 (as shown in FIG. 6B or FIG. 5B) is attached to the container 118 to enclose the second section 38″ of the reservoir 22″. The dispensing cap 106 may attach via threads 102, 90′, or may attach via a snap fit or other suitable means. The bag separation member 30″ may snap or thread or slip onto to the container 118 or dispensing cap 106, or may be clamped between the container and dispensing cap. A breast pump as shown in FIG. 5C may be used to fill the bag 30″, either separate from the container 118 or with the container. The solution reservoir could alternately be equipped with a non-luer port for attachment to a syringe to fill the solution reservoir without removing the cap, making filling easier. solution reservoir may also have an air vent for removing air from the reservoir when priming the reservoir. The vent may be a hydrophobic filter which does not allow liquid to pass through the vent.

Once the bag 30″ is filled with milk, vitamins or other additives may be added to the milk. Alternatively, any feeding solution may be placed in the bag 30″. The prepared and filled solution reservoir 22″ is used in the manner discussed above. As pump fluid is introduced into the first section 34″ of the solution reservoir 22″, the bag separation member 30″ is collapsed and the feeding solution is expelled from the second section 38″ of the solution reservoir.

The solution reservoirs 22′, 22″ shown in FIGS. 5A and 6A are advantageous in that they allow for complete or near complete dispensing of the feeding solution from the second section of the solution reservoir. These solution reservoirs 22′, 22″ are also advantageous in that they may be used in combination with a standard breast pump 98 to allow a mother to pump milk directly into the second section 38′, 38″ of the reservoir, eliminating the need to transfer the milk to another container for dispensing. Eliminating the need for transferring milk to another container is beneficial as it reduces the likelihood for spilling, contaminating, or otherwise harming the milk. Eliminating the need for transferring the milk also eliminates waste, as some fluid is usually lost when transferring between containers.

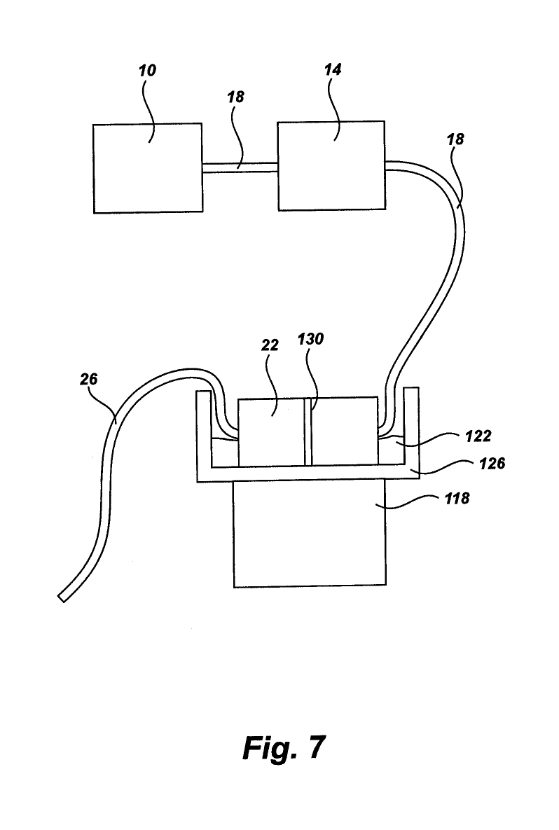

Turning now to FIG. 7, another schematic view of the feeding system of the present invention is shown. The feeding system is the same as has been shown in FIG. 1 with the introduction of an additional feeding solution conditioner. As such FIG. 7 encompasses the details of the individual pieces of the system as shown in FIGS. 2 through 6. The solution reservoir 22 has been placed in a conditioner 118. The conditioner 118 may perform various functions according to the particular needs. The conditioner may be a heater or chiller to keep the feeding solution at a desired temperature. Additionally, the conditioner 118 may be a agitator or mixer for preventing the feeding solution from settling during use. The conditioner 118 may both agitate the feeding solution and keep the feeding solution at a desired temperature during feeding.

It is appreciated that, especially with prenatal infants where a slow feeding rate is required, it is possible for the feeding solution to separate into the various components or fall outside of a desired temperature range. The use of a conditioner 118 is therefore desirable in some situations. The conditioner may hold liquid such as water 122 in a pan 126, and may heat or cool the pan 126 to thereby heat or cool the feeding solution. The water 122 may not be necessary, but may help in providing even heating or cooling. The conditioner 118 may agitate the solution reservoir 22 by rocking from side to side or by vibrating. Either may be accomplished by pivotably attaching the pan 126 to the conditioner 118 and by further attaching the pan 126 to an off-center hub on a drive wheel via a connecting rod and driving the drive wheel with a motor at a desired speed. It may or may not be necessary or desirable to attach the solution reservoir 22 to the conditioner 118, but such may easily be accomplished with a strap 130 or other clamp.

It is appreciated that, because the solution reservoir is separate from the pump 14, it is much easier to heat, cool, or agitate the contents of the solution reservoir. It is often impractical or impossible to control the temperature or mix the contents of a pump where the feeding solution is carried on a reservoir on the pump.

As has been discussed, the system may use a variety of different means for introducing pump fluid into the feeding solution reservoir. Many different types of pumps are available which are suitable for use with the present system. Additionally, other means are suitable. For example, a gravity driven system may be used to introduce pump fluid into the feeding solution reservoir. Such a system may use an IV bag of saline and a drip chamber to regulate the flow rate of the liquid, and thus the flow rate of the feeding solution. In such a situation, it may be desirable to use a reservoir which does not require a large amount of pressure to realize flow. Other pumping means may include a static pressure cuff. It is generally desirable that the pumping device is capable of providing a pump fluid to the reservoir at a controlled rate.

The present application discusses the use of the present system in the context of providing feeding solution to a person such as a premature or neo-natal infant. It is appreciated that the present system could also be used to provide a variety of supplements, nutritional solutions, medications, IV solutions, etc. to a person. The present system is desirable in situations which benefit from the advantages of the present invention, such as a reduction in lost fluid, where mixing or temperature regulation is desirable, etc.

There is thus disclosed an improved solution delivery system. It will be appreciated that numerous changes may be made to the present invention without departing from the scope of the claims.

What is claimed is:

1. A method for delivering a medical solution comprising:
selecting a solution reservoir having a separation member which divides the solution reservoir into a first section and a second section;
placing a medical solution in the second section of the solution reservoir;
introducing a pump fluid into the first section of the solution reservoir to thereby displace solution from the second section of the solution reservoir and thereby deliver the medical solution to a patient.

2. The method for delivering a medical solution to a patient of claim 1, wherein the medical solution comprises breast milk.

3. The method for delivering a medical solution to a patient of claim 1, wherein the medical solution is a feeding solution.

4. The method for delivering a medical solution to a patient of claim 1, wherein the medical solution is an IV solution.

5. The method for delivering a medical solution to a patient of claim 1, wherein the medical solution comprises medicine.

6. The method for delivering a medical solution to a patient of claim 1, wherein the method further comprises selecting a solution reservoir having a separation member which is slidably disposed between the first section and the second section, and wherein the method comprises using the pump fluid to force the separation member to slide and thereby expel medical solution from the solution reservoir.

7. The method for delivering a medical solution to a patient of claim 1, wherein the method further comprises selecting a solution reservoir having a separation member which is flexible.

8. The method for delivering a medical solution to a patient of claim 1, wherein the method further comprises attaching the second section of the solution reservoir to a patient tube.

9. The method for delivering a medical solution to a patient of claim 1, wherein the method further comprises pumping the pump fluid into the first section of the solution reservoir with a pump.

10. The method for delivering a medical solution to a patient of claim 1, wherein the method comprises delivering a proportionately smaller amount of medical solution to the patient than pump fluid delivered into the solution reservoir.

11. A method of delivering a medical solution comprising:
disposing a medical solution in a solution reservoir disposed in fluid communication with a patient; and
introducing a pump fluid different than the medical fluid into the solution reservoir to dispense medical solution out of the solution reservoir and into a patient while maintaining the pump fluid separate from the medical solution.

12. The method according to claim 11, wherein the method comprises using a peristaltic pump to introduce pump fluid into the solution reservoir.

13. The method according to claim 11, wherein the method comprises using a syringe pump to introduce pump fluid into the reservoir.

14. The method according to claim 11, wherein the method comprises dispensing medical solution at a rate proportional to but different than a rate at which the pump fluid is introduced into the solution reservoir.

15. The method according to claim 11, wherein the method comprises attaching the solution reservoir to a breast pump and disposing breast milk therein.

16. The method according to claim 11, comprising maintaining separation between the pump fluid and the medical solution with a movable separation member.

17. A method for delivering feeding solution to a patient, the method comprising:
selecting a solution reservoir having movable separation member which divides the solution reservoir into a first reservoir section and a second reservoir section;
filling the second reservoir section with a feeding solution; and
introducing a pump fluid into the first reservoir section so as to move the movable separation member and thereby dispense the feeding solution from the second reservoir section.

18. The method according to claim 17, wherein the method comprises using breast milk as the feeding solution.

19. The method according to claim 18, further comprising attaching the solution reservoir to a breast pump.
